(19) United States
(12) Patent Application Publication
(10) Pub. No.: US 2016/0106541 A1
(43) Pub. Date: Apr. 21, 2016

(54) Prosthesis Component With Antimicrobially Coating Sliding Surface

(71) Applicant: WALDEMAR LINK GMBH & CO. KG, Hamburg (DE)

(72) Inventor: Roger Thull, Hamburg (DE)

(73) Assignee: WALDEMAR LINK GMBH & CO. KG, HAMBURG (DE)

(21) Appl. No.: 14/833,861

(22) Filed: Aug. 24, 2015

Related U.S. Application Data
(63) Continuation of application No. 13/253,597, filed on Oct. 5, 2011, now abandoned.
(60) Provisional application No. 61/424,292, filed on Dec. 17, 2010.

(30) Foreign Application Priority Data
Oct. 5, 2010 (EP) 10013320.6

(51) Int. Cl. A61F 2/30; A61F 2/38; A61F 2/32; A61L 27/06; A61L 27/30

(52) U.S. Cl. CPC A61F 2/30767; A61L 27/06; A61L 27/306; A61F 2/32; A61F 2/38; A61F 2/3094; A61L 2430/24; A61F 2002/3098; A61F 2310/0052; A61F 2310/00856; A61F 2310/0097

(57) ABSTRACT

In a prosthesis component of a joint endoprosthesis, the prosthesis component comprises a sliding surface that is designed to form a sliding joint with a counter sliding surface of another prosthesis component. The sliding surface may be formed by a sliding surface coating deposited on the body of the prosthesis component, wherein the sliding surface coating has an antimicrobial effect. The sliding surface coating is active against microorganisms in the area of the sliding joint.

### CROSS REFERENCE TO RELATED APPLICATIONS

The present application claims the benefit of European Application Serial No. 10013320.6 filed 5 Oct. 2010 and U.S. Provisional Application Ser. No. 61/424,292 filed 17 Dec. 2010; both of which are incorporated herein by reference in their entireties.

### FIELD OF THE INVENTION

The invention relates to a prosthesis component of a joint endoprosthesis. The prosthesis component comprises a sliding surface that is designed to form a sliding joint with a counter sliding surface. The counter sliding surface can be a surface of another prosthesis component or a bony counter bearing of the joint or soft tissue.

### BACKGROUND

When prostheses are inserted into the human body, repeatedly problems arise because the body does not accept the prosthesis without complication. Frequently, so-called biofilms that form after the colonization of the surface with bacteria, also in the implanted state, are the reason for complications. It has been shown that there is not only a risk associated with the surface regions of the prosthesis that in the implanted state abut directly against bodily tissue, but also with such surface regions that are intended to interact with other prosthesis components. These surface regions include, in particular, the sliding surfaces of a prosthesis component that interact with other prosthesis components in order to reproduce the functionality of the natural joint. Surface biofilms in these regions are largely beyond reach of the defense mechanisms of the body. The risk exists that biofilms repeatedly release microorganisms from the area of the sliding surfaces into the surrounding regions. The tendency of the microorganisms to distribute themselves from the area of the sliding surfaces into the surrounding regions is further enhanced by the fact that the sliding surfaces are continuously subject to movement.

The sliding surfaces of the prostheses are subject to quite different stresses than the remaining surface regions. Where applicable, relative movements are taking place in the joint while almost the entire weight of the human body multiplied by acceleration forces in function rests on the sliding surfaces of the prosthesis. Under these circumstances it is difficult to prevent spreading of the microorganisms.

### SUMMARY

In view of this prior art the problem underlying the invention is to present a prosthesis component for which the probability is reduced that complications occur during insertion into the human body. The problem is solved by the features of claim 1. According to the invention, the prosthesis component is provided with a sliding surface coating which exhibits an antimicrobial efficacy. Advantageous embodiments are described in the dependent claims.

Initially a few terms are explained. A coating with antimicrobial efficacy distinguishes itself by the fact that it has the potential to lower the viability or reproduction capability of microorganisms. Commonly this is accomplished by the coating releasing a substance that has an efficacy against microorganisms.

The sliding joint of a prosthesis is formed by means of a sliding surface of a first prosthesis component and a corresponding counter sliding surface of a second prosthesis component. During the course of a movement the contact between sliding surface and counter sliding surface can be made in varying areas of the sliding surface. The term sliding surface encompasses all surface areas that can come in contact with the counter sliding surface during joint movements in the prosthesis.

If a coating is deposited on a body, additional material is added to that body. On the surface of the body a coating is created that consists entirely of newly added material. It is not excluded that the coating according to the invention my also be deposited on surface areas of the prosthesis component other than the sliding surface. Frequently, however, the other surface areas are free of the coating so that the coating is only on the sliding surface.

With the coating according to the invention the antimicrobial effect is introduced in the area of the sliding surfaces and therefore in an area that is not accessible to the body's defense system. Without antimicrobial activity in the area of the sliding surfaces microorganisms can establish themselves there and multiply again and again. The antimicrobial coating according to the invention acts against the microorganisms and thereby reduces the risk of complications.

Antimicrobial coatings, as such, on the surface of prostheses are known, see for example EP 2 036 517. They were up until now however only intended for such surface areas of the prosthesis that are exposed to low mechanical stresses.

An antimicrobial agent that is particularly suitable for application in the body is silver. Silver particles that are released from a coating have a good efficacy against microorganisms that are situated on the surface of the coating. If the silver particles emerge from the coating without encountering microorganisms they combine in the body electrolyte to silver chloride (AgCl) and can be excreted from the body in this form. The silver then has no damaging effect on other body cells. The sliding surface coating therefore comprises preferably silver. The silver is released from the sliding surface coating preferably in the form of individual silver ions.

The sliding surfaces of a joint prosthesis are exposed to high mechanical stresses. A high durability of the sliding surfaces can be achieved by making the sliding surface coating a hard material coating. Preferably the sliding surface coating comprises a nitride, an oxynitride, or an oxide on the basis of a refractory metal. Advantageously, silver is also contained in the sliding surface coating and can be released from the coating. Refractory metals are high melting point, non-precious metals of the fourth, fifth and sixth sub group. Encompassed in the fourth sub group are titanium, zirconium and hafnium, in the fifth sub group vanadium, niobium and tantalum, and in the sixth sub group chromium, molybdenum and wolfram. Refractory metals particularly suitable for the coating of endoprosthesis components are titanium, zirconium, niobium and tantalum. The term nitride, oxynitride or oxide on the basis of a refractory metal refers to compounds that the ions of a refractory metal form with oxygen and/or nitrogen as a reactive gas. These compounds distinguish themselves though great hardness. If the sliding surface coating additionally comprises silver, then not only ions of the refractory metal and a reactive gas are involved in the creation of the nitride, oxynitride or oxide, but in addition also silver ions. The silver ions are integrated into the coating that is being created.

In the case of a hard material coating that contains silver, the antimicrobial efficacy increases with the content of silver. If one were to design the coating primarily with high antimicrobial efficacy in mind, one would select a content of silver in the hard material coating of about 25% by weight. However, the mechanical durability is then markedly lowered in comparison with a hard material coating that does not contain silver. In the context of the invention a lower silver content by weight in the hard material coating should therefore be selected. The content by weight is preferably between 2% and 15%, more preferably between 3% and 10%. It has been shown that with such a low silver content a good compromise between mechanical durability and antimicrobial effect is achieved.

The sliding surface coating should be formulated such that the silver is released primarily in the form of individual silver ions and not in the form of larger particles. Individual silver ions can be active between the sliding surfaces without developing an abrasive effect with respect to the sliding surfaces.

The body of the prosthesis component can consist of metal or a metal alloy. Particularly suitable materials are titanium and titanium alloys.

A sliding surface coating according to the invention can, for example, be deposited on the body of the prosthesis component by means of a PVD-process (Physical Vapor Deposition). To this end, targets are provided from which the material that later is to form the coating can be removed. An individual target can be provided that comprises the refractory metal as well as the silver. It is, however, also possible to provide several targets, wherein a first target comprises a refractory metal and wherein a second target comprises silver.

For the purpose of removing the ions, for example an electric arc can be generated between an electrode and the target, which delivers to the target enough energy locally so that ions are removed. Another possibility of delivering sufficient energy locally to the target may consist of aiming a laser beam at the target. Alternatively, even if very expensive, the energy delivery by means of an electron beam is also possible.

The free ions are conducted onto the body of the prosthesis component. For this purpose a voltage can be applied between the target and the body by means of which the ions are accelerated in the direction of the body. The reaction vessel with target and body contains a reactive gas, such as for example oxygen or nitrogen or a mixture of oxygen and nitrogen. The ions of the refractory metal as well as the ions of silver and/or copper react with the reactive gas. The sliding surface coating according to the invention forms by means of the reaction that is occurring on the surface of the body. The reaction can take place under vacuum, preferably under high vacuum.

If formation of the coating on other surface areas of the prosthesis components besides the sliding surface is to be avoided, these surface areas can be covered during the coating process. For example, these surface areas can be covered with a temperature stable material. A material is labelled as temperature stable if it is stable under the conditions present during plasma coating processes. The temperature stable material can in particular be a metal.

In order to keep the abrasion during movements between the sliding surface and the counter sliding surface to a minimum, the sliding surface should be as smooth as possible. Preferably the average roughness Rₐ of the sliding surface as determined according to DIN EN ISO 4288 and 3274 is smaller than 0.05 μm.

The invention further relates to a joint endoprosthesis with a first prosthesis component designed according to the invention and a second prosthesis component. The second prosthesis component comprises a counter sliding surface which forms a sliding joint with the sliding surface of the first prosthesis component.

In what follows, the invention is described in reference to the enclosed drawings by example of an advantageous embodiment.

### BRIEF DESCRIPTION OF THE DRAWINGS

The invention is described by example in what follows in reference to the enclosed drawings.

FIG. 1 a hip prosthesis equipped as per the invention;

FIG. 2 an enlarged section from FIG. 1; and

FIG. 3 a knee prosthesis equipped as per the invention.

### DETAILED DESCRIPTION OF THE INVENTION

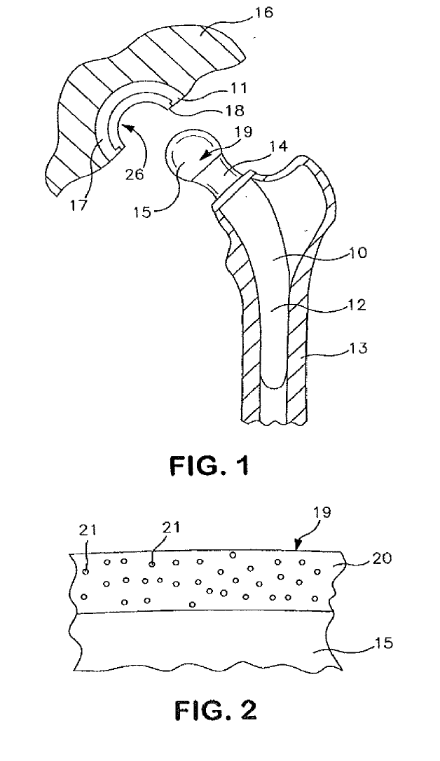

A hip prosthesis shown in FIG. 1 comprises a femur component 10 and an acetabulum component 11. The femur component 10 comprises a shaft 12 that is inserted into the marrow space of a femur 13. Following the shaft 12 is a prosthesis neck 14 that transitions into a joint head 15. The acetabulum component 11 is introduced into the hip bone 16 and replaces there the natural acetabulum. The acetabulum component 11 comprises a shell 17 that is made of metal as well as an insert 18 consisting of high-density polyethylene.

In the implanted state the joint head 15 lies in the polyethylene insert 18 in such a manner that joint head 15 together with the polyethylene insert 18 forms a sliding joint. The surface of the joint head 15 thereby acts as a sliding surface 19 together with a counter sliding surface 26 of the polyethylene insert 18. The average roughness Rₐ of the sliding surface is smaller than 0.03 μm.

The femur component 10 is manufactured from a titanium alloy. In the area of the sliding surface 19, as FIG. 2 shows in a significantly enlarged representation, a sliding surface coating 20 is deposited on the joint head 15. The sliding surface coating 20 is manufactured by means of a PVD-process and has a thickness of about 3 μm. The sliding surface coating 20 consists of titanium nitride with enclosed silver atoms 21, wherein the silver atoms are not represented to scale.

The sliding surface coating 20 has a silver atom content by weight of about 5%. The silver is contained in the sliding surface coating 20 in the form of individual atoms and not in the form of larger particles. During utilization of the prosthesis individual silver ions emerge from the sliding surface coating 20. The silver ions have an antimicrobial effect in the area of the sliding joint. The individual silver ions are so small that they have no abrasive effect on the polyethylene insert 18.

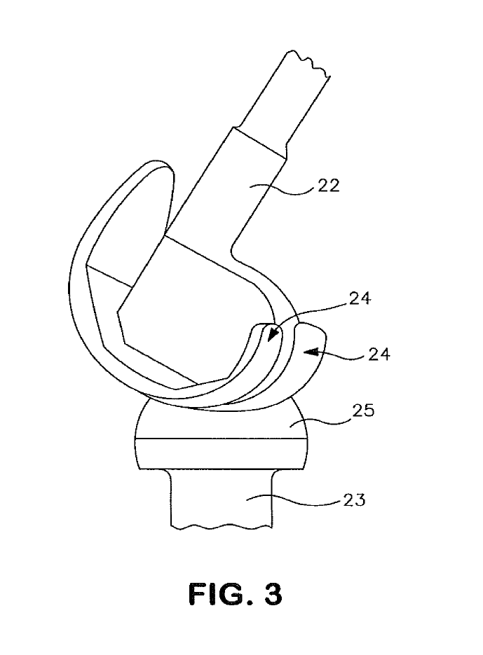

In FIG. 3 a knee prosthesis is shown that is equipped with a sliding surface coating according to the invention. The knee prosthesis comprises a femur component 22 and a tibia component 23. Sliding surfaces 24 of the femur component 22 act together with a polyethylene insert 25 of the tibia component 23 and form a sliding joint that simulates the function of the natural knee.

The femur component 22 and the tibia component 23 consist of a classic chromium-cobalt alloy. An antimicrobial sliding surface coating 20, as it is illustrated in FIG. 2, is deposited on the sliding surfaces 24 of the femur component 22.

In the case of the knee prosthesis of FIG. 3 only a small surface area of the sliding surfaces 24 lies at any point in time on top of the polyethylene insert 25. The respective surface area changes according to the state of motion of the knee prosthesis. The sliding surface coating 20 extends over the entire sliding surfaces 24, meaning over all surface areas that can come in contact with the polyethylene insert 25. The silver ions emerging from the sliding surface coating 20 have an antimicrobial effect on the microorganisms situated there.

1-21. (canceled)

22. A joint endoprosthesis comprising:
a prosthetic component manufactured from a titanium alloy, the prosthetic component having a joint head with a sliding surface;
a sliding surface coating of titanium nitride with enclosed silver atoms on the sliding surface, the average roughness of the sliding surface being smaller than 0.03 μm; and
an insert made of polymer for receiving the joint head to form a sliding joint;
wherein, during use, individual silver atoms emerge from the sliding surface coating.

23. The endoprosthesis of claim 22, wherein the silver content by weight in the sliding surface coating is between 2% and 15%.

24. The joint endoprosthesis of claim 22, wherein the silver content by weight in the sliding surface coating is between 3% and 10%.

25. The joint endoprosthesis of claim 22, further comprising a sliver atom content of the coating of about 5% by weight.

26. The joint endoprosthesis of claim 22, wherein the sliding surface coating has a thickness of between 0.5 μm and 5 μm.

27. The joint endoprosthesis of claim 22, wherein the sliding surface coating has a thickness of approximately 3 μm.

28. The joint endoprosthesis of claim 22, wherein the sliding surface coating is obtainable by a PVD-process.

29. A knee prosthesis comprising:
a prosthetic component manufactured of a chromium-cobalt alloy, the prosthetic component having sliding surfaces;
a sliding surface coating of titanium nitride with enclosed silver atoms on the sliding surfaces, the average roughness of the sliding surface being smaller than 0.03 μm; and
an insert made of polymer for receiving the sliding surfaces, the sliding surfaces contacting the insert;
wherein, during use, individual silver atoms emerge from the coating.

30. The knee prosthesis of claim 29, wherein the silver content by weight in the sliding surface coating is between 2% and 15%.

31. The knee prosthesis of claim 29, wherein the silver content by weight in the sliding surface coating is between 3% and 10%.

32. The knee prosthesis of claim 29, further comprising a sliver atom content of the coating of about 5% by weight.

33. The knee prosthesis of claim 29, wherein the sliding surface coating has a thickness of between 0.5 μm and 5 μm.

34. The knee prosthesis of claim 29, wherein the sliding surface coating has a thickness of approximately 3 μm.

35. The knee prosthesis of claim 29, wherein the sliding surface coating is obtainable by a PVD-process.
